(19) United States
(12) Patent Application Publication
(10) Pub. No.: US 2014/0100501 A1
(43) Pub. Date: Apr. 10, 2014

(54) Hyperextension Brace

(71) Applicants: Steven Burke, Huntington Beach, CA (US); Geoffrey Garth, Long Beach, CA (US); Erik Zimmer, Oceanside, CA (US)

(72) Inventors: Steven Burke, Huntington Beach, CA (US); Geoffrey Garth, Long Beach, CA (US); Erik Zimmer, Oceanside, CA (US)

(73) Assignee: ASPEN MEDICAL PARTNERS, LLC, Irvine, CA (US)

(21) Appl. No.: 14/054,624

(22) Filed: Oct. 15, 2013

Related U.S. Application Data
(63) Continuation-in-part of application No. 12/977,039, filed on Dec. 22, 2010, now Pat. No. 8,556,840.
(60) Provisional application No. 61/289,247, filed on Dec. 22, 2009.

(51) Int. Cl. A61F 5/02

(52) U.S. Cl. CPC A61F 5/026
USPC 602/19

(57) ABSTRACT

A posture correction brace for a person has a tightening mechanism that allows a user to pull a cord to pull down on the back of a pair of shoulder straps, thereby pulling the shoulders back to correct the person's posture. The front ends of the shoulder straps are typically also attached to the back of the wearer, to help the posture correction brace pull the shoulders back as the shoulder straps tighten.

This application is a continuation-in-part of U.S. utility application having Ser. No. 12/977039, filed on Dec. 22, 2010, which claims priority to U.S. provisional application having Ser. No. 61/289247, filed on Dec. 22, 2009. These and all other extrinsic materials discussed herein are incorporated by reference in their entirety. Where a definition or use of a term in an incorporated reference is inconsistent or contrary to the definition of that term provided herein, the definition of that term provided herein applies and the definition of that term in the reference does not apply.

### FIELD OF THE INVENTION

The field of the invention is orthotics for posture correction.

### BACKGROUND

The following background discussion includes information that may be useful in understanding the present invention. It is not an admission that any of the information provided herein is prior art or relevant to the presently claimed invention, or that any publication specifically or implicitly referenced is prior art.

Existing braces are limited in that there is no one-size fits all. Users with different height and torso widths need different sizes. There needs to be a brace that can be used for different users and yet still provide a tight, comfort, and proper fit to the user's body.

U.S. Pat. No. 5,548,843 to Chase et al. teaches a support belt in which the user can pull the belt straps to secure the shoulders. However, only the front portion of Chase's shoulder straps can be shortened, which tends to pull the shoulders forward instead of back. This hurts the wearer's posture, and could be extremely detrimental where the user has a medical issue with his posture, for instance where the wearer has a thoracic kyphosis.

WIPO publ. no. 2009/052031 to Sandifer et at (the '031 brace) teaches a brace where a user can pull “fastening pads” around the waist to tighten the abdominal and at the same time tighten the user's shoulder straps. However, the '031 brace is not adjustable for different users, and specifically teaches that in order for the brace to fit a different user than the user for which it is designed, a physician would have to cut the straps. The '031 brace also teaches a pair of shoulder straps attached to a spinal frame member via rivets around the midsection of the post, which is rigid along the spinal frame member.

WIPO publ. no. 2009/017499 to Sandifer et at (the '499 brace) teaches a brace with a spinal frame member that can be adjusted along with the shoulder strap. However, the '499 brace requires the spinal frame to be adjusted via screws in pre-drilled holes and is not movable once the screws are in place. If the spinal frame needs to be adjusted, the brace must first be removed from the user and then the screws need to be adjusted. If the spinal frame needs to be adjusted to a position in between the pre-drilled holes, a new hole would need to be drilled or a new spinal frame created.

U.S. Pat. No. 5,954,250 to Hall also teaches a harness with an adjustable spinal frame, however, like Sandifer, Hall's spinal frame must be adjusted by another person, or when the wearer is not wearing Hall's harness.

Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

Thus, there is still a need in the art for improved adjustable braces that help correct a wearer's posture.

### SUMMARY OF THE INVENTION

The inventive subject matter provides apparatus, systems and methods in which a posture correction brace pulls the shoulders back of a person with a pull cord that inferiorly adjusts a posterior portion of each of the straps along a person's back when the cord is pulled. As used herein, the term “cord” means any device flexible at room temperature that mechanically pulls a device attached to one end of the cord with a device attached to another end of the cord, for example a rope, belt, monofilament, braid, or string. As used herein, the term “inferiorly adjusts” means to adjust a device downwards from the person's head towards the person's feet. As used herein, the term “posterior” means any part of the body behind the midline of the person, and preferably along the rear surface of the person (which excludes the side surfaces which start along the surface of the person's arms or ears. As used herein, the term “along the person's back” means that the strap is pulled within a 45 degree angle along the wearer's midline, and more preferably within a 30 degree or a 15 degree angle along the wearer's midline.

Generally, the two posterior portions of the shoulder straps couple to a latch on the back of the wear that is pulled down as the pull cord is pulled, although the pull cord itself could attach to each posterior portion of the shoulder straps separately without departing from the scope of the invention. Preferably, the ends of the shoulder straps are attached to a hinge, or the shoulder straps comprise a single strap that threads through a loop to form two separate shoulder straps. As used herein, a “loop” is any structure that a cord could be threaded through to change a pulling direction of the cord, such as a hole, hook, pulley, or corner. Shoulder straps could be made from any suitable material, for example leather, vinyl, rubber), and is preferably flexible. The shoulder strap could have padding coupled to a shoulder area of the strap to help prevent chafing while the strap is adjusted inferiorly.

The brace is preferably configured such that the pull cord could be pulled forward by the wearer while the brace is worn, which would then pull back on the shoulder straps, thereby pulling back on the wearer's shoulders as the pull cord is drawn. For example, one end of the cord could be coupled to the shoulder straps, and the cord could be threaded orthogonally through a loop to run forward along the waist of the user. This would allow the user to pull one end of the cord anteriorly forward, which would then translate that force to pull the other end of the cord inferiorly downward along the back.

The pull cord and the posterior portion of each of the shoulder straps could be coupled via a slider that rides along a vertical rail to ensure that the shoulder straps are adjusted approximately parallel to the wearer's midline. The slider could have a second tightening mechanism that ensures that the pull cord remains taught after the shoulder straps are pulled in place. Alternatively or in addition to the tightening mechanism, the rail could comprise matching indents or detents that match the slider to allow a user to “click” through a plurality of adjustment positions. In an exemplary embodiment, the slider comprises a base and an adjustable extender that allows a wearer to first adjust a range of movement for the posterior portions of the shoulder straps before adjusting the height of the posterior portions of the shoulder straps.

Preferably an anterior portion of the pull cord, such as the end of the pull cord, is coupled to a pull tab that is easily grasped by the wearer. In an exemplary embodiment, the pull tab is coupled to a belt around the waist of the wearer for easy access, for example using a hook and loop fastener, a button, a pocket, or a magnetic coupling on the belt. Anterior portions of the shoulder straps, such as the anterior ends of the shoulder straps, could be coupled to a posterior portion of the belt, which would further assist in drawing the wearer's shoulders back as the posterior portions of the shoulder strap are tightened.

The belt generally wraps around the wearer, preferably about the lumbar area. An exemplary belt has a stiff portion around the lumbar area of the wearer to support the lumbar area of the wearer. The belt could also have an adjustment mechanism that tightens the belt around the wearer, such as a strap threaded through a loop, a cloth tie, or overlapping hook and loop anterior ends. Preferably, the adjustment mechanism comprises one or more pulley systems that tighten the belt around the wearer, for example around the lumbar area of the wearer, the lateral area of the wearer, or evenly around the wearer. A second pull cord could be coupled to the belt's adjustment mechanism such that the second pull cord could be pulled forward by the wearer to tighten the adjustment mechanism while the brace is worn. In an exemplary embodiment, the first and second pull cords are positioned on opposite sides of the wearer, such that the wearer could pull forward on both pull cords simultaneously to both pull the shoulders back and to pull the belt taught.

The length of the shoulder straps is preferably also adjustable to accommodate wearers of different heights. Additionally, anterior portions of the shoulder straps could be pulled towards one another using a chest strap or buckle, providing for a snug fit.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIGS. 1A-1B are rear perspective views of an embodiment of the current inventive subject matter with the shoulder straps comprising a single strap wrapped around both shoulders of the wearer.

FIG. 2 shows a front perspective view of an alternative embodiment of the current inventive subject matter.

FIG. 3 shows a rear perspective view of the alternative embodiment in FIG. 2.

FIGS. 4A-4B show a perspective and an exploded view of the slider used in the alternative embodiment of FIG. 2.

FIGS. 5A-5B show perspective views of the alternative embodiment of FIG. 2 with a wearer adjusting a circumferential pressure.

FIGS. 6A-6B show perspective views of the alternative embodiment of FIG. 2 with a wearer adjusting the length of the shoulder straps

FIGS. 7A-7D show perspective views of the alternative embodiment of FIG. 2 with a wearer adjusting the posterior portion of each of the shoulder straps.

FIG. 8A shows a perspective view of a schematic of another embodiment of a posture correction brace having a biasing element in a retracted position.

FIG. 8B shows an enlarged, perspective view of the brace of FIG. 8A.

FIG. 8C shows a perspective view of the brace of FIG. 8A having the biasing element in an extended position.

### DETAILED DESCRIPTION

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

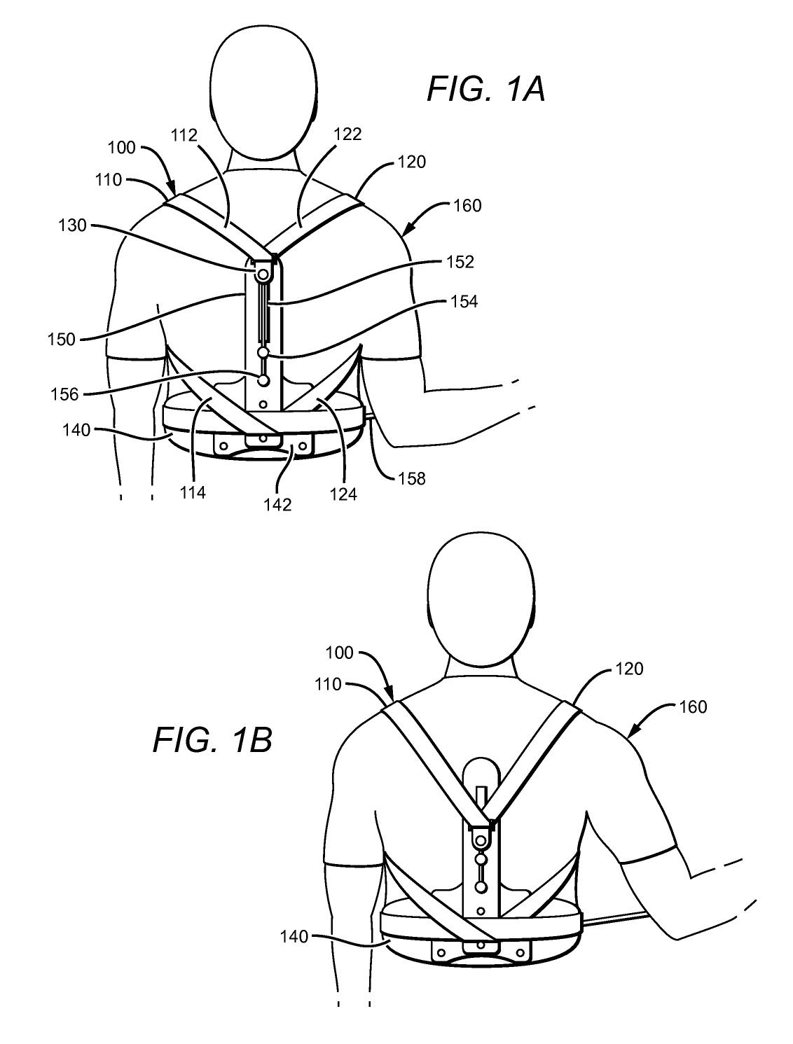

In FIGS. 1A-1B, an exemplary posture correction brace 100 has a pair of shoulder straps 110 and 120 coupled to a hinge 130 that is joined to a belt 140 via slider 150. While shoulder straps 110 and 120 are coupled to hinge 130 using a thin hole, the shoulder straps could be coupled to the hinge using any suitable loop. Shoulder straps 110 and 120 comprise a single strap that divides into two separate components through hinge 130. Shoulder straps 110 and 120 have superior sections 112 and 122, respectively and inferior sections 114 and 124, respectively. Superior sections 112 and 122 are coupled to hinge 130, which draws those sections inferiorly along wearer 160's back as hinge 130 is drawn downwards along slider 150. Inferior sections 114 and 124 are coupled to posterior portion 142, and then around the waist of wearer 160. The anterior ends (not shown) of straps 110 and 120 then meet at the front of wearer 160, such that wearer 160 could then tighten belt 140 by manipulating the anterior ends, for example by simply tying the ends together or by threading the ends through a loop.

As shown, slider 150 comprises a rail 152 along which hinge 130 slides. Hinge 130 is sized and dimensioned to slide towards or away from pulleys 154 and 156 when cord 158 is pulled or loosened, respectively. One end of cord 158 is coupled to hinge 130, and cord 158 threads through pulleys 154 and 156 such that the other end of cord 158 rests on a side of the wearer. This allows the wearer to draw hinge 130 downwards along slider 150 by pulling forward on cord 158 with a pull-tab. FIG. 1A depicts slider 150 in its superior-most position when cord 158 is loosened, while FIG. 1B depicts slider 150 in its inferior-most position when cord 158 is pulled taught.

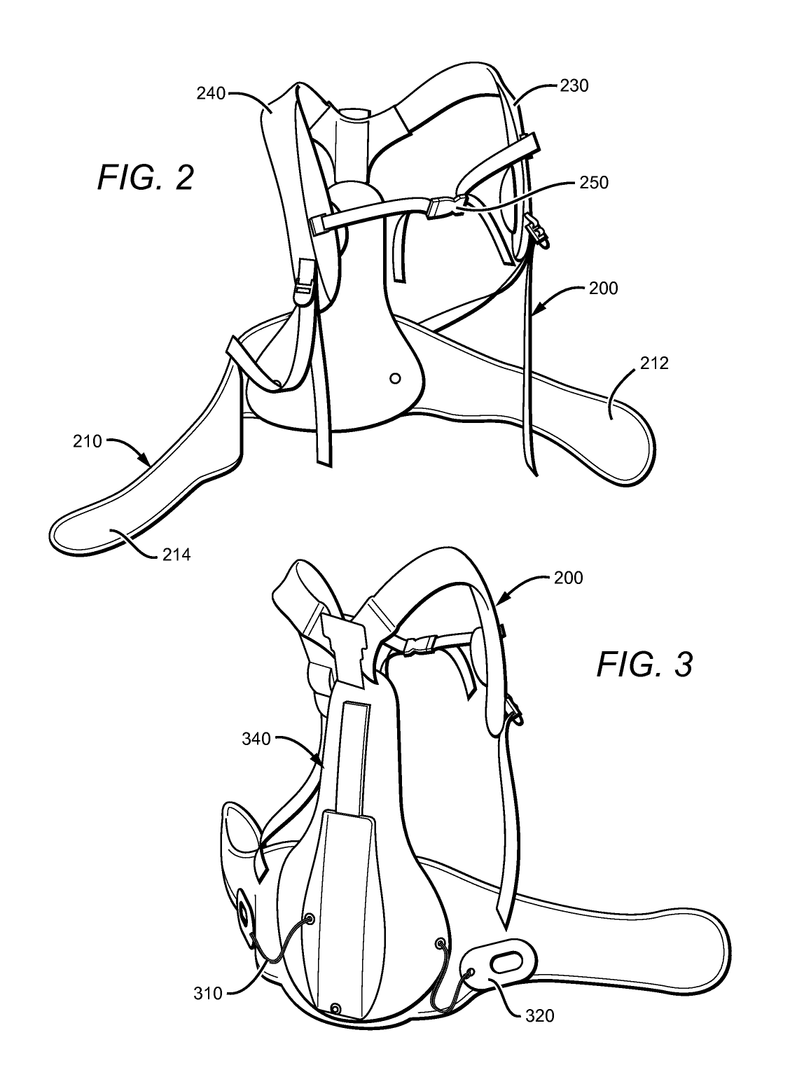

In FIG. 2, an alternative posture correction brace 200 has belt 210, left shoulder strap 230, right shoulder strap 240, and chest buckle 250. FIG. 3 shows a rear view of brace 200 with left cord 310, right cord 320, and slider 340.

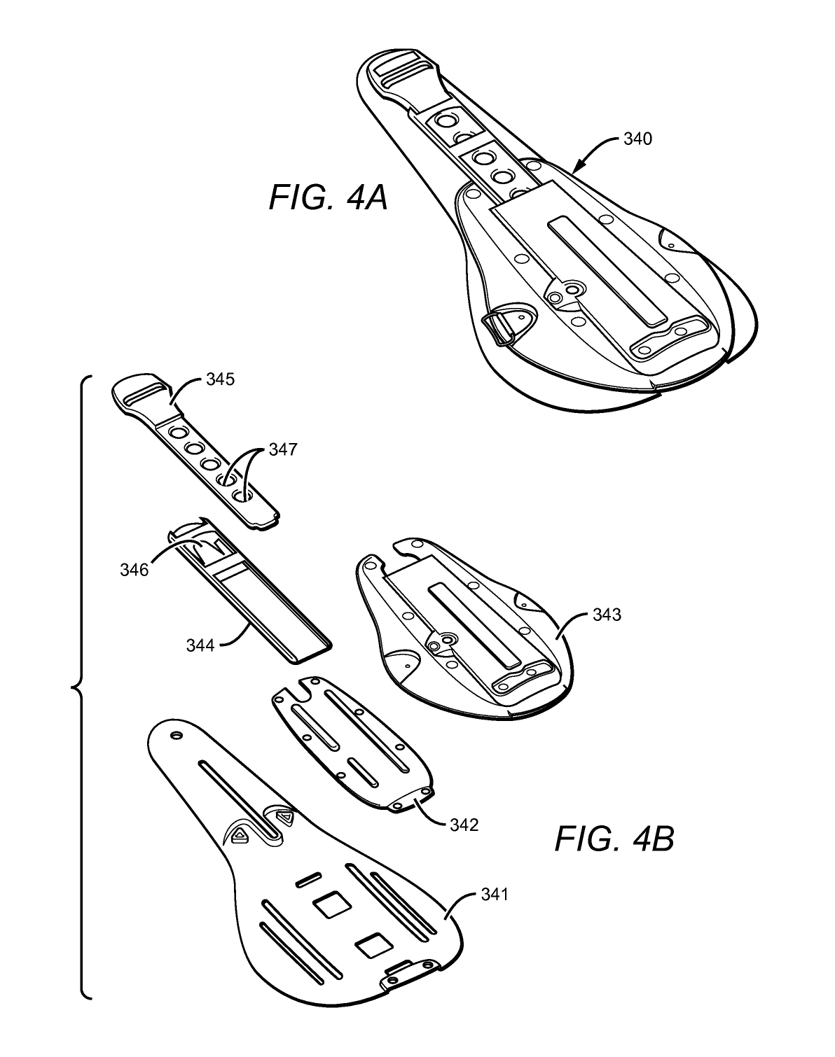

FIGS. 4A and B is an exploded view of slider 340, with base panel 341, track base 342, track cover 343, sheath 344, and extender 345. Sheath 344 couples to extension 345 by threading sheath detent 346 through extender holes 347. By threading sheath detent 346 through different extender holes 347, a user effectively adjusts a range of movement for posterior portions of left shoulder strap 230 and right shoulder strap 240, since both are attached to the superior end of extender 345. After the extender 345 length is adjusted, the extender 345 could be inserted into track base 342 which is then held against the track by sandwiching base panel 341 against track cover 343, which both effectively limit a travel of the extender 345 approximately to the length of sheath 344.

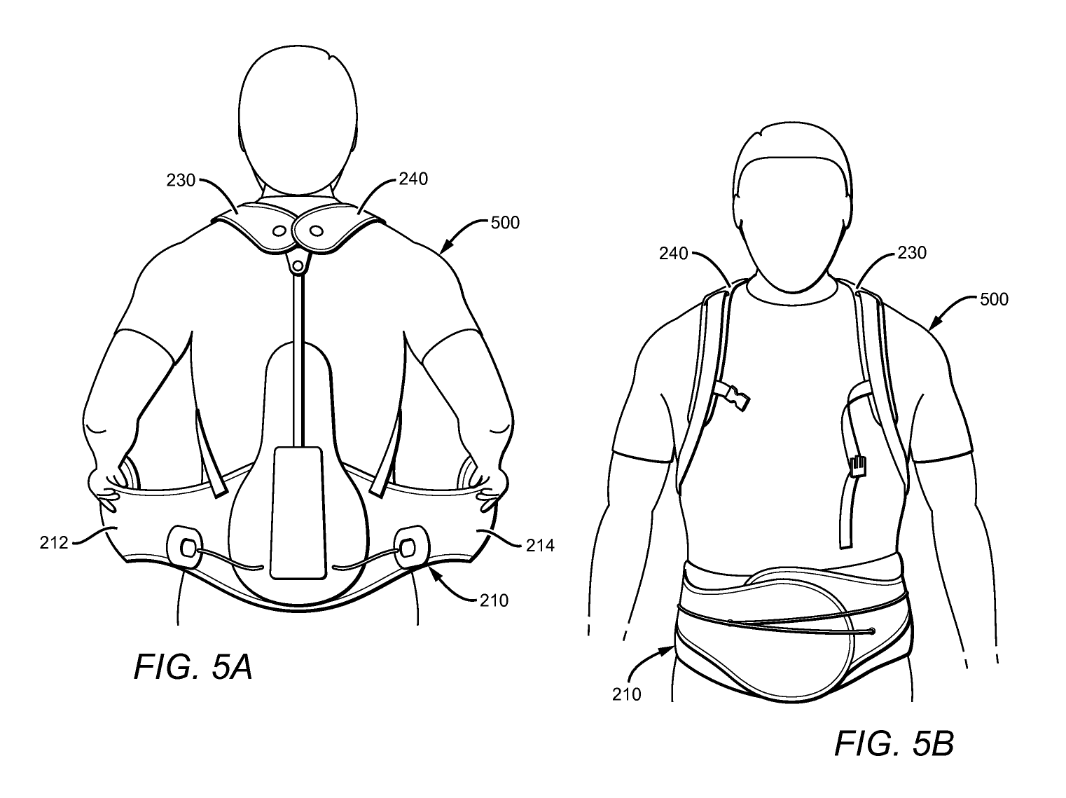

FIGS. 5A-5B show perspective views of a wearer 500 putting on brace 200 and adjusting belt 210. First, wearer 500 threads his left and right arms through the left shoulder strap 230 and right shoulder strap 240, respectively. Belt 210 has left anterior portion 212 and right anterior portion 214 that overlap one another around the stomach of wearer 500 to provide a snug fit, such as in copending application Ser. No. 12/394867, which is incorporated herein by reference.

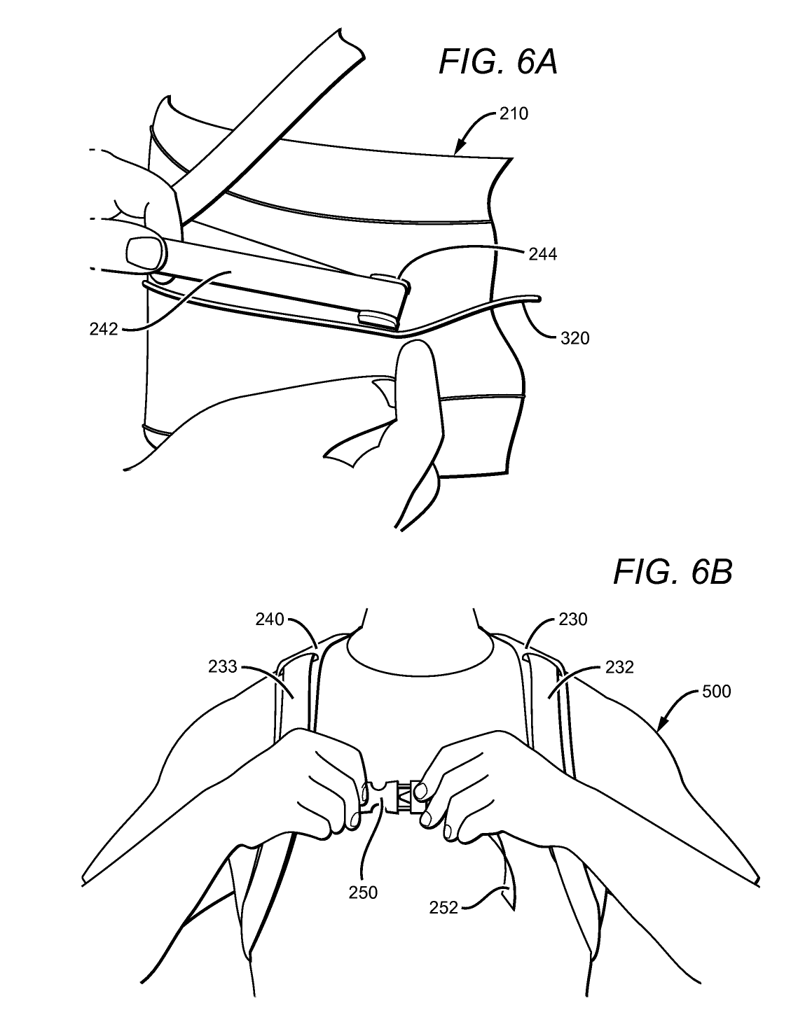

FIGS. 6A-6B show perspective views of wearer 500 then adjusting left shoulder strap 230 and right shoulder strap 240 about wearer 500. Anterior portion 242 of right shoulder strap 240 couples to the anterior portion of belt 210 via loop clasp 244, which could be opened to allow a user to lengthen or shorten the effective length of left shoulder strap 230. Wearer 500 could then tighten the shoulder straps using chest buckle 250. Chest buckle 250 has a strap 252 that could be used to draw anterior portion 232 of left shoulder strap 230 towards anterior portion 242 of right shoulder strap 240, creating a snug fit.

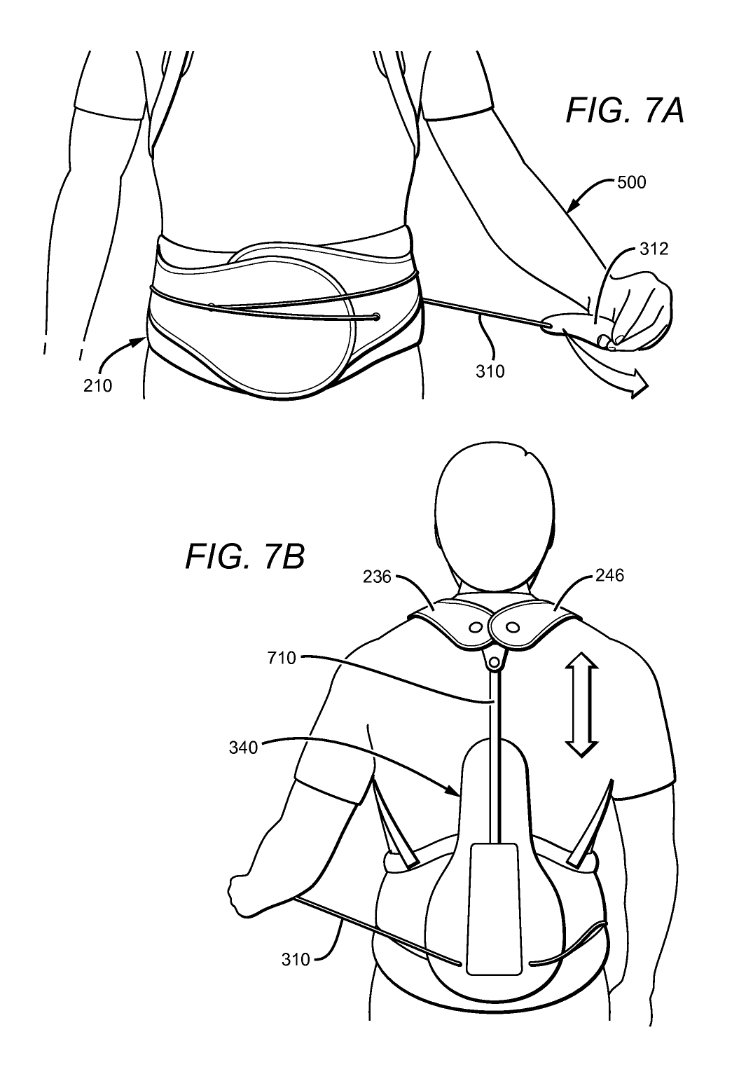

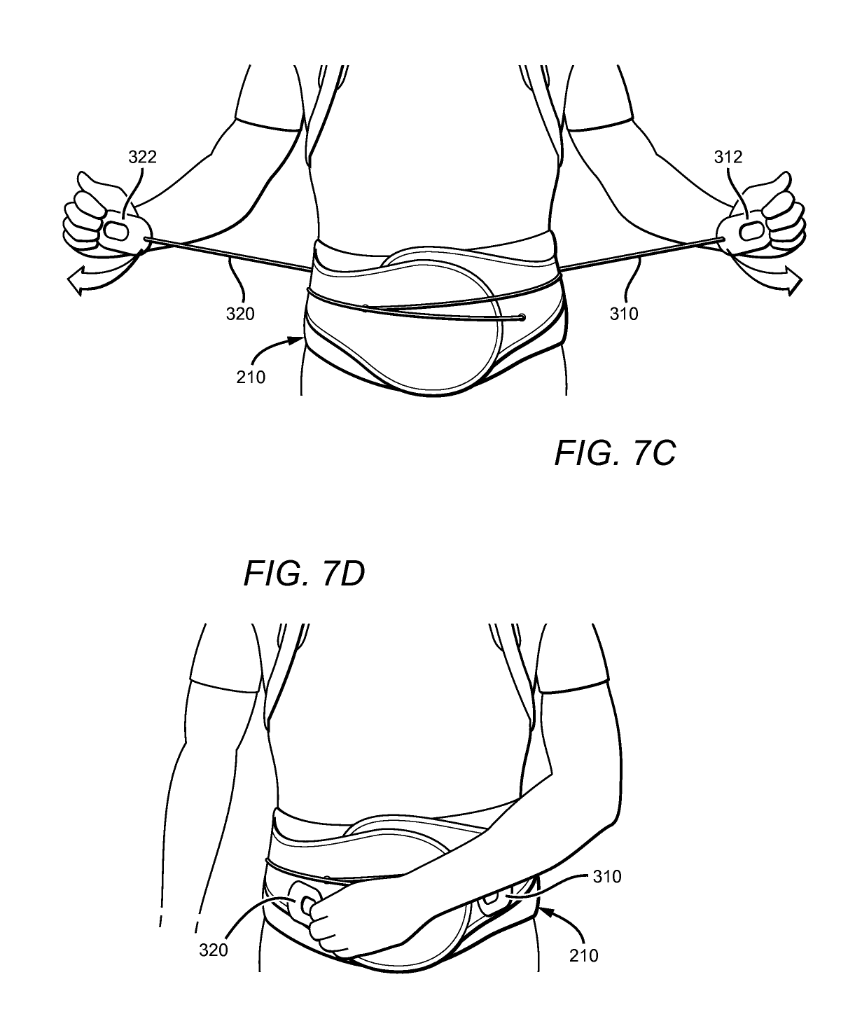

FIGS. 7A-7D show perspective views of wearer 500 inferiorly adjusting posterior portion 246 of right shoulder strap and posterior portion 236 of left shoulder strap 230 inferiorly along wearer 500's back. Since posterior portion 236 of left shoulder strap 230 and posterior portion 246 of right shoulder strap 240 are coupled to slider 340 using strap 710, as wearer 500 pulls left cord 310, extender 345 is pulled down along track base 342 within slider 340. This effectively pulls the shoulders of wearer 500 back, correcting the posture of wearer 500 and holding the shoulders back.

Right cord 320 is coupled to a lumbar adjustment mechanism similar to that disclosed in copending application Ser. No. 12/394867, which allows a user to adjust both belt 210 and slider 340 by pulling on right cord 320 and left cord 310, respectively, as shown in FIG. 7C. Pull tabs 312 and 322 are then coupled to the front of belt 210 using hook and loop fasteners. While pull tabs 312 and 322 are coupled to the belt using standard hook and loop fasteners, pull tabs 312 and 322 could be coupled to the belt in any other suitable manner without departing from the scope of the invention.

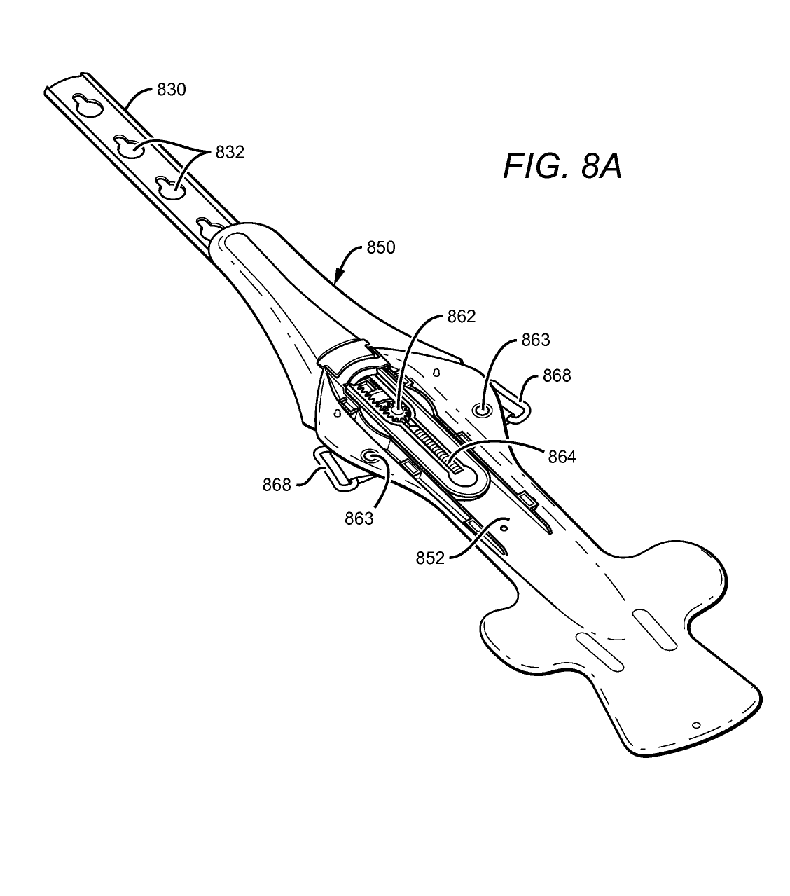

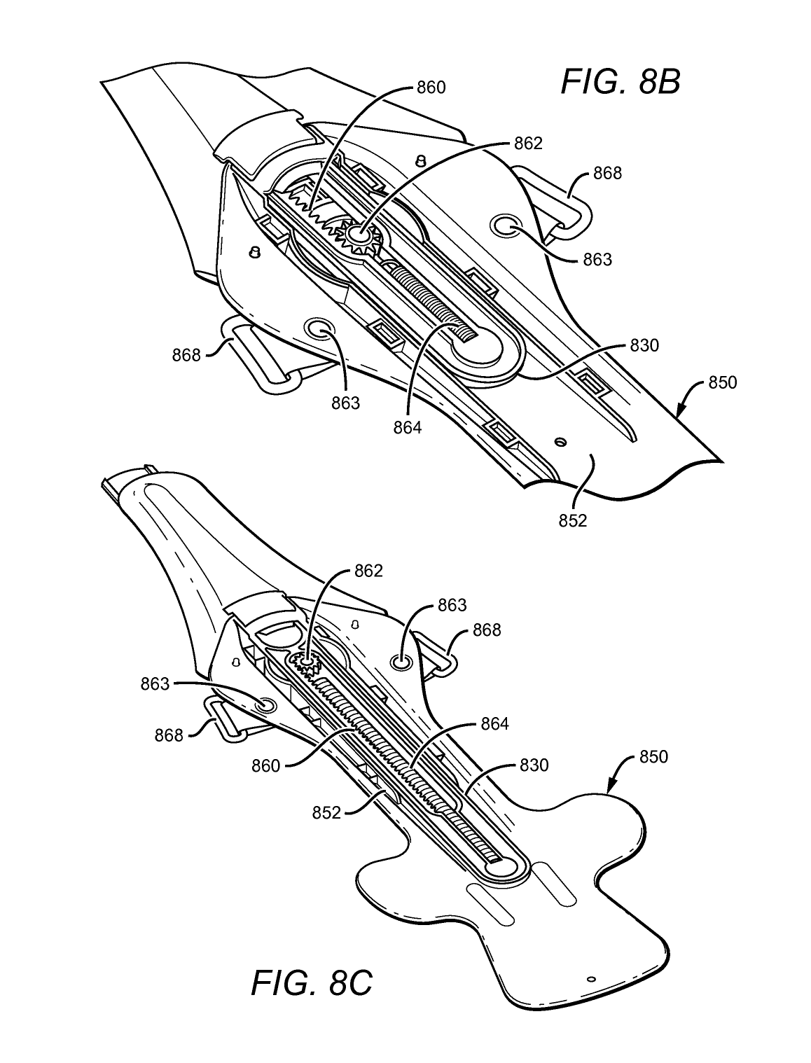

FIGS. 8A-8C illustrate another embodiment of a slider 850 having a guide 852 that maintains an orientation of arm 830 with respect to the slider. As shown, guide 852 can comprise two walls extending outwardly from the slider 850, such that the arm 830 can move in an inferior or superior direction with a wall on either side of the slider 850. Of course, guide 852 could be any commercially suitable form such that the orientation of the arm 830 is maintained with respect to the slider 850.

It is contemplated that the slider 850 can include a rack 860 and pinion 862, with the arm 830 having a rack 860 either attached thereto or that comprises a portion of the arm 830. The slider 850 can comprise a pinion 862 that cooperates with the teeth of the rack 860. This advantageously allows a position of the arm 830 with respect to the slider 850 to be locked by preventing rotation of the pinion 862 and thereby preventing movement of the rack 860 and arm 830. In addition, the rack and pinion can help to maintain a horizontal position of the arm 830 with respect to slider 850. Although shown with the pinion 862 disposed within a hollow portion of the arm 830, it is also contemplated that the pinion 862 could be disposed adjacent to the arm 830.

Slider 850 can further include one or more biasing elements. As shown in FIGS. 8A-8C, slider can include a spring 864 that couples the arm 830 to the slider 850 and that biases the arm 830 in a superior direction. Of course, any commercially suitable element could be used that provides sufficient force to bias the arm 830 in a superior direction. For example, in FIG. 8A, the spring 864 is in a retracted position when the arm 830 is maximum superior position with respect to slider 850. In contrast, FIG. 8C illustrates the arm in a maximum inferior position with the spring 864 in an extended position. In such position, spring 864 comprises potential energy that will cause the arm 830 to move in a superior direction such that the arm 830 can return to the position shown in FIG. 8A. In this manner, the arm 830 and the shoulder straps that attach to the arm 830 such as shown in FIG. 1A, for example, will return to an initial position with the arm 830 and shoulder straps in at upmost superior position with respect to slider 850.

Arm 830 can be coupled to shoulder straps either directly or indirectly such as via a hinge as discussed above. It is contemplated that the shoulder straps can be coupled to the arm 830 via one or more apertures 832 in arm 830, which allow for variable placement of the shoulder straps with respect to the arm 830 thereby facilitating adjustment of the slider 850, and ultimately the brace that includes the slider 850, to differently sized patients. In such embodiments, when the arm 830 moves in an inferior direction, for example, the shoulder straps, and in particular, a posterior portion of the shoulder straps, can be pulled in the inferior direction as well.

In embodiments where the brace includes a cord that inferiorly adjusts a posterior portion of each of the pair of shoulder straps along the person's back when the cord is pulled, such as those described above, it is contemplated that the arm 830 can also be moved in an inferior direction as the cord is pulled. This in turn causes each shoulder strap to also move in the inferior direction where the shoulder straps are coupled to the arm 830. By including a biasing element such as spring 864, the arm 830 and shoulder straps will move in a superior direction with the cord is released due to the potential energy in the extended spring until the spring 864 reaches a retracted or semi-retracted position.

Slider 850 can further include loops 868 to which one or more belts or straps can be coupled such as by threading a portion of the belt or strap through the loop. Preferably, the loops 868 can be coupled to the slider by a rivet 863 or other means, such that the loops 868 can pivot with respect to the slider 850.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A posture correction brace for a person, comprising:
a pair of shoulder straps;
a pull cord that inferiorly adjusts a posterior portion of each of the pair of shoulder straps along the person's back when the cord is pulled; and
a biasing element configured to bias the pair of shoulder straps superiorly with respect to the person.

2. The brace of claim 1, further comprising a belt that extends around the person.

3. The brace of claim 2, wherein the pull cord extends from the belt.

4. The brace of claim 2, further comprising a second pull cord that tightens the belt around the person.

5. The brace of claim 4, further comprising a pulley system that tightens the belt around a lumbar region of the person.

6. The brace of claim 1, wherein the pull cord adjusts the posterior portion of each of the pair of shoulder straps through a slider that rides along a rail.

7. The brace of claim 6, wherein the slider comprises a base and an adjustable extender.

8. The brace of claim 1, further comprising a chest strap that pulls an anterior portion of each of the pair of shoulder straps towards one another.

9. The brace of claim 2, further comprising a pull-tab coupled with the pull cord, wherein the pull-tab is attachable to the belt using a hook and loop fastener.

10. The brace of claim 2, wherein an anterior portion of each of the pair of shoulder straps couples to a posterior portion of the belt.

11. The brace of claim 1, wherein a length of at least one of the pair of shoulder straps is adjustable.

12. The brace of claim 1, wherein the pull cord is threaded orthogonally to allow the person to adjust the posterior portion of each of the pair of shoulder straps inferiorly by pulling the pull cord anteriorly.

13. A posture correction brace for a wearer, comprising:
left and right shoulder straps, each of which has an anterior and posterior portion;
a slider coupled to the left and right shoulder straps, and configured to move in a superior or inferior direction along a rail; and
a biasing element coupled to the slider and configured to bias the slider in a superior position with respect to a wearer.

14. The brace of claim 13, wherein the biasing element comprises a spring.

15. The brace of claim 13, further comprising a cord that inferiorly adjusts a position of the slider inferiorly when the cord is pulled.

16. The brace of claim 15, wherein the biasing element is further configured to cause the slider to move in a superior direction when the cord is released.

17. The brace of claim 13, further comprising a belt that extends around the wearer.

18. The brace of claim 13, wherein a posterior portion of each of the left and right shoulder straps is adjusted as the slider moves in the superior or inferior direction.

19. A posture correction brace for a person, comprising:
a pair of shoulder straps;
a cord coupled to the pair of shoulder straps, wherein the cord inferiorly adjusts a posterior portion of the shoulder straps along the person's back in an inferior direction when the cord is pulled; and
a biasing element coupled to the pair of shoulder straps and configured to cause the posterior portion of the shoulder straps to move in a superior direction along the person's back when the cord is released.
